(19) United States
(12) Patent Application Publication
(10) Pub. No.: US 2014/0358011 A1
(43) Pub. Date: Dec. 4, 2014

(54) SINGLE SHOT HIGH RESOLUTION CONJUNCTIVAL SMALL VESSEL PERFUSION METHOD FOR EVALUATING MICROVASCULATURE IN SYSTEMIC AND OCULAR VASCULAR DISEASES

(71) Applicant: University of Miami, Miami, FL (US)

(72) Inventors: Hong JIANG, Miami, FL (US); Delia DeBUC, Pembroke Pines, FL (US); Jianhua WANG, Miami, FL (US)

(73) Assignee: University of Miami, Miami, FL (US)

(21) Appl. No.: 14/288,774

(22) Filed: May 28, 2014

Related U.S. Application Data
(60) Provisional application No. 61/828,388, filed on May 29, 2013.

(51) Int. Cl. G06T 7/00; A61B 3/14

(52) U.S. Cl. CPC G06T 7/0012; A61B 3/145; G06T 2207/30041
USPC 600/476; 382/128

(57) ABSTRACT

A system and method for generating high-resolution, small vessel perfusion maps (nCPMs) of the ocular conjunctival microvasculature. Unlike current systems and methods, the present invention allows for the generation of nCPMs using only a single raw image obtained in a single image acquisition step. The method includes obtaining a single raw image of at least a portion of the microvasculature of the ocular conjunctiva. The raw image may be obtained using a digital camera and slit lamp. The red and blue color channels are removed from the raw image to create a color-adjusted image in which contrast between the microvasculature and the sclera is enhanced. The color-adjusted image is then inverted and saved as a grayscale inverted image. The grayscale inverted image may be skeletonized. The grayscale image and/or the skeletonized image may be quantitatively analyzed.

### CROSS-REFERENCE TO RELATED APPLICATION

This application is related to and claims priority to U.S. Provisional Patent Application Ser. No. 61/828,388, filed May 29, 2013, entitled SINGLE SHOT HIGH RESOLUTION CONJUNCTIVAL SMALL VESSEL PERFUSION METHOD FOR EVALUATING MICROVASCULATURE IN SYSTEMIC AND OCULAR VASCULAR DISEASES, the entirety of which is incorporated herein by reference.

### STATEMENT REGARDING FEDERALLY SPONSORED RESEARCH OR DEVELOPMENT

n/a

### FIELD OF THE INVENTION

The present invention relates to a method and system for generating high-resolution small vessel perfusion maps of the ocular or bulbar conjunctiva using a single raw image.

### BACKGROUND OF THE INVENTION

The conjunctiva, composed of non-keratinized, stratified columnar epithelium with interspersed goblet cells, is disposed on the inside of the eyelids and covers the sclera. The conjunctiva is involved in mucus and tear production as well as providing a protective barrier against pathogens. Microcirculation within the conjunctiva can serve as the window to the body, and may be ideal for the evaluation of pathologic conditions or diseases that affect systemic and ocular circulation. For example, conjunctival microcirculation characteristics may be used for the evaluation of diabetes and associated conditions.

The vasculature in the eye and vasculature of the cerebral cortex have the same main blood supply, which is the internal carotid artery (ICA). The bulbar or ocular conjunctiva covers the eyeball, over the anterior sclera. Bulbar conjunctival vasculature is regarded as the terminal vascular bed of the human ICA, and these vessels can be accessed directly and non-invasively. Some studies of the conjunctival microvasculature have provided sensitive indicators of both systemic vascular diseases and vascular diseases of the central nervous system (CNS). Further, bulbar conjunctival blood flow is sensitive to changes of the supplying vessels. For example, bulbar conjunctival blood flow has been shown to correlate to cerebral blood flow during aortic arch surgery, and has also been used for intraoperative monitoring during carotid artery surgery. In one study, microcirculation was attenuated when the ICA was clamped and was restored immediately after ICA reperfusion. In other studies, conjunctival microangiopathy was reported in type I diabetic patients that was reversed following simultaneous pancreas-kidney transplantation, and sickle cell-related conjunctival microangiopathy was reported that improved following poloxamer 188 treatments. Still other studies have demonstrated the use of conjunctiva microcirculation as an indicator of cerebral microcirculation and hemodynamics in patients with diabetes. For example, diabetic conjunctival microangiopathy was identified earlier than diabetic retinopathy.

Current methods of imaging and evaluating conjunctival microcirculation include fractal analysis and related vascular-based quantitative measurements of the branching patterns of the ocular microvasculature. However, these methods have not demonstrated high sensitivity or specificity due to, at least in part, a lack of high resolution microvasculature maps. For example, in one study, a Heidelberg Retinal Flowmeter (HRF; Heidelberg Engineering, Heidelberg, Germany) was modified to measure bulbar conjunctival blood flow, but only arbitrary results were provided. Other studies used orthogonal polarized spectral imaging to measure conjunctival capillary perfusion in a single vessel; however, this procedure was limited by the very small image field. Still other studies used a computer-assisted video camera to analyze conjunctival microcirculation with specially developed software. Up to fifteen minutes of recording was typically required, and the field was approximately 8 mm². High-speed video microcinematography (MSN) has also been used to measure pre-capillary arteriole blood flow velocity, including the minimum of the end diastolic values and the maximum of all of the peak systolic values and their average. However, this method required a manual approach to the registration of sequential images, which was time consuming. Improvements to this method include a semi-automated registration method was used on images of the human conjunctiva using a sequence of 62 images obtained with a Zeiss slit-lamp biomicroscope equipped with a digital charged couple device camera, but the image-to-geometry registration problem still requires the user to verify the correctness of the registration. Therefore, user bias is still a limitation since certain key operations rely on the user to guide the registration.

Recently, retinal capillary perfusion maps have been obtained with an advanced imaging modality called Retinal Function Imager (RFI, Optical Imaging Ltd, Rehovot, Israel). RFI is a fundus camera-based device attached to a specific camera (a 60-Hz, 1024×1024-pixel digital camera) that captures reflectance changes as a function of time under stroboscopic illumination (wavelengths between 530 and 590 nm). Using this method, eight consecutive flashes with an interflash interval of less than 20 ms are used to take eight images. Hemoglobin in red blood cells acts as an intrinsic motion-contrast agent in the generation of detailed noninvasive capillary-perfusion maps (nCPMs), and the blood flow velocity is calculated using the proprietary software of the device. However, multiple sets of images are needed to generate the perfusion maps of the retina. When this method is applied to the conjunctiva, it is found that the resulting small vessel perfusion map is not as clear as the retinal perfusion map. Because of the blurred images of the conjunctival perfusion maps obtained with RFI, it was more difficult to do image segmentation for quantitative analysis using commercial software like the Fractal Analysis Toolbox.

It is therefore desired to provide a method for generating a high-resolution small vessel perfusion map of the conjunctiva in a single “shot” (or image acquisition step), useful for predicting and evaluating systemic and ocular vascular diseases.

### SUMMARY OF THE INVENTION

The present invention advantageously provides a method and system for generating high-resolution small vessel perfusion maps of the ocular or bulbar conjunctiva using a single raw image. In one embodiment, a method for generating a small vessel perfusion map of an ocular conjunctiva may include obtaining a single raw image of at least a portion of microvasculature of the ocular conjunctiva, the raw image including at least a red color channel, a blue color channel, and a green color channel; removing the red color channel and the blue color channel from the raw image to create a color-adjusted image of the microvasculature; and inverting the color-adjusted image to create a grayscale inverted image of the microvasculature. The raw image may be obtained using a camera. The red color channel and the blue color channel may be removed using image processing software. The method may further include skeletonizing the grayscale inverted image of the microvasculature to create a skeletonized image of the microvasculature. For example, “skeletonizing” may include digitally reducing anatomical boundaries of one or more of the plurality of vessels to one or more linear shapes, each linear shape having a uniform diameter. The grayscale inverted image of the microvasculature may be cropped to include a region of interest. The region of interest may be skeletonized and the microvasculature of the skeletonized image may be evaluated using fractal analysis (for example, multifractal analysis). Further, at least one of the grayscale inverted image and the skeletonized image may be quantitatively analyzed. Still further, microvascular density may be evaluated using at least one of the grayscale inverted image and the skeletonized image. The method may further include determining a threshold value for at least one of conjunctival artery diameter and conjunctival vein diameter; determining from at least one of the grayscale inverted image and the skeletonized image an average value for at least one of conjunctival artery diameter and conjunctival vein diameter; and comparing the at least one threshold value to the at least one average value. The method may further include determining the presence of a medical condition based at least in part on the comparison, or a response of the ocular conjunctiva to at least one of contact lens wear and the use of an ophthalmic solution may be evaluated based at least in part on the comparison. The medical condition may be at least one of cerebral small vessel disease, dry eye, chronic inflammation, multiple sclerosis, ocular stress, and hypoperfusion. Additionally or alternatively, the medical condition may be at least one of allergies, ocular tumors, corneal transplant rejection, glaucoma, hypertension, diabetes, vascular dementia, Parkinson's disease, and Alzheimer's disease.

In one embodiment, a method of evaluating microvasculature of an eye may include obtaining a single raw image of at least a portion of microvasculature of the ocular conjunctiva, the image including at least a red color channel, a blue color channel, and a green color channel; removing the red color channel and the blue color channel from the raw image to create a color-adjusted image of the microvasculature; inverting the color-adjusted image to create a grayscale inverted image of the microvasculature; and skeletonizing the grayscale inverted image of the microvasculature to create a skeletonized image of the microvasculature, the microvasculature including a plurality of vessels, and skeletonizing the grayscale inverted image including digitally reducing one or more of the plurality of vessels to one or more linear shapes, each linear shape having a uniform diameter.

In one embodiment, a system for generating a small vessel perfusion map of an ocular conjunctiva may include a slit lamp; and a computer in electrical communication with the slit lamp, the computer including one or more processors programmed to: obtain a single raw image of at least a portion of microvasculature of the ocular conjunctiva from the slit lamp, the raw image including at least a red color channel, a blue color channel, and a green color channel; remove the red color channel and the blue color channel to create a grayscale image of the microvasculature; invert the grayscale image; and calculate at least one conjunctival microvasculature characteristic. The at least one conjunctival microvasculature characteristic may be selected from the group consisting of: average conjunctival vein diameter, and average conjunctival artery diameter. The microvasculature may include a plurality of vessels and the one or more processors may be further programmed to skeletonize the grayscale inverted image to create a skeletonized image and perform a fractal analysis on the skeletonized image. Additionally, the one or more processors may be further programmed to determine a threshold value for at the least one conjunctival microvasculature characteristic, compare the at least one threshold value to the at least one calculated microvasculature characteristic, and determine the presence of a medical condition based at least in part on the comparison.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a simplified cross-sectional view of an exemplary human eye;

FIG. 2 shows a non-limiting, exemplary system for obtaining single-shot, high-resolution small vessel perfusion maps of the conjunctival microvasculature;

FIGS. 3A-3E show a method for obtaining single-shot, high-resolution small vessel perfusion maps of the conjunctival microvasculature;

FIG. 3A shows a raw image of the conjunctiva;

FIG. 3B shows the raw image of FIG. 3A with color adjustments to improve contrast;

FIG. 3C shows a grayscale inversion (capillary perfusion map, or nCPM) of the adjusted image of FIG. 3B;

FIG. 3D shows a close-up view of a region of interest of the inverted image of FIG. 3C;

FIG. 3E shows a skeletonized view of the region of interest of FIG. 3D for fractal analysis;

FIGS. 4A-4C show non-limiting, exemplary graphs generated using software used for quantitative analysis of the small vessel perfusion map created as a result of the steps of FIGS. 3A-3E;

FIG. 5 shows an exemplary capillary perfusion map generated with the Retinal Function Imager (RFI) currently used in the art. The image was created from multiple shots and shows a blurred image of vessels;

FIG. 6 shows a raw image, capillary perfusion map, and skeletonized view thereof for exemplary normal patients, and associated quantitative and multifractal analysis results;

FIG. 6 shows an exemplary chart of fractal analysis values during and after contact lens use;

FIG. 7 shows a comparison of raw images of a human eye before and after using phenylephrine hydrochloride ophthalmic solution; and

FIG. 8 shows a comparison of raw images and nCPMs of a normal human eye and a human eye of a subject having cerebral small vessel disease (CSVD).

### DETAILED DESCRIPTION OF THE INVENTION

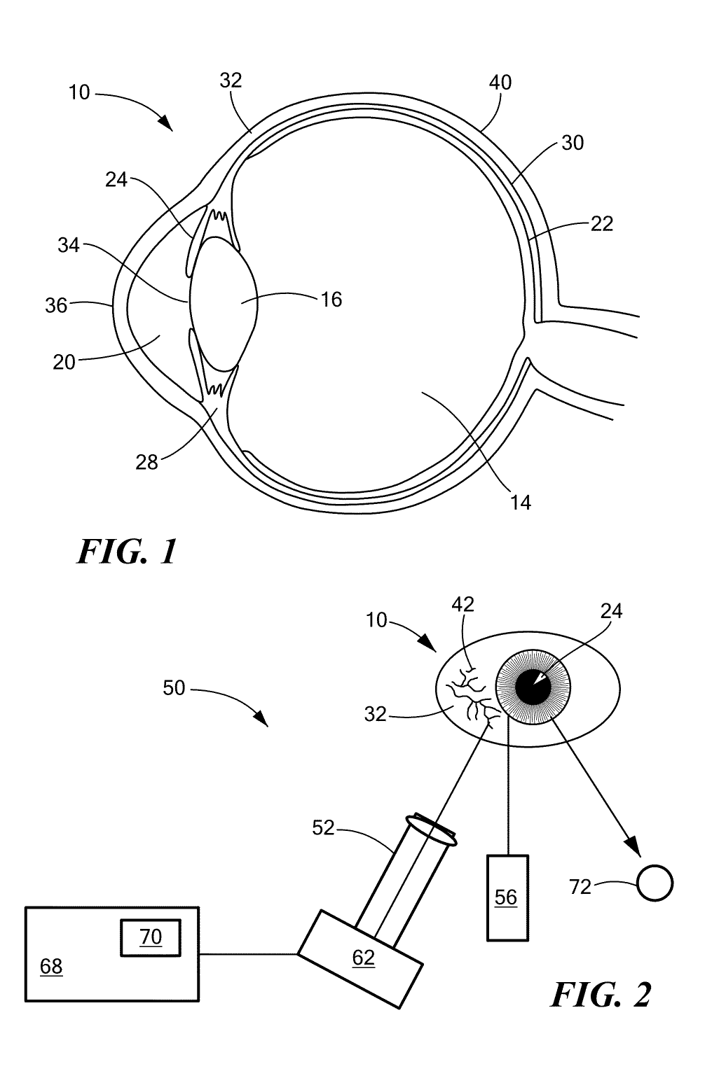

The present invention advantageously provides a method and system for generating high-resolution small vessel perfusion maps of the ocular or bulbar conjunctiva using a single raw image. Referring now to the drawing figures in which like reference designations refer to like elements, a cross-sectional view of a human eye 10 is shown in FIG. 1. The eye 10 generally includes a vitreous body 14, lens 16, aqueous humour 20, retina 22, iris 24, ciliary body 28, choroid 30, and the sclera 32. The aqueous humour 20, the iris 24, and the pupil 34 are covered by a transparent cornea 36, and the sclera 32, the white part of the eye, is covered by a conjunctiva 40. The conjunctiva 40 includes an epithelial layer, which contains, among other things, blood vessels 42 (which may be referred to herein as a “capillary bed”). The blood vessels 42 of the conjunctiva 40 may be visible against the white or whitish sclera 32 behind.

Referring now to FIG. 2, a non-limiting, exemplary system 50 for obtaining single-shot, high-resolution small vessel perfusion maps of the conjunctival microvasculature is shown. The system 50 may generally include a slit lamp 52 of the type that is commonly used for ophthalmic imaging. The slit lamp 52 may include a high-intensity light source 56 that can be focused and directed onto the eye 10. Further, the slit lamp 52 may be used in connection with an image capture device 62, such as a standard still and/or video camera or a biomicroscope. In the exemplary system 50 shown in FIG. 2, the slit lamp 52 may be used with an image capture device 62 such as a digital camera (for example, Canon EOS 7D 18.0 megapixel digital SLR camera) and a high-magnification lens (for example, Volk 60D lens). The slit lamp 52 and/or the image capture device 62 may be in electrical communication with a computer 68 having one or more processors 70 for the acquisition and processing of data received from the slit lamp 52 and/or the image capture device 62, as described in more detail below. It will be understood that other high-resolution imaging devices may also be used, such as a scanning laser opthalmoscope. The system 50 may also include a fixation target 72, which may be any point at which the subject focuses his or her gaze. This may help reduce movement in the target eye 10 during image acquisition.

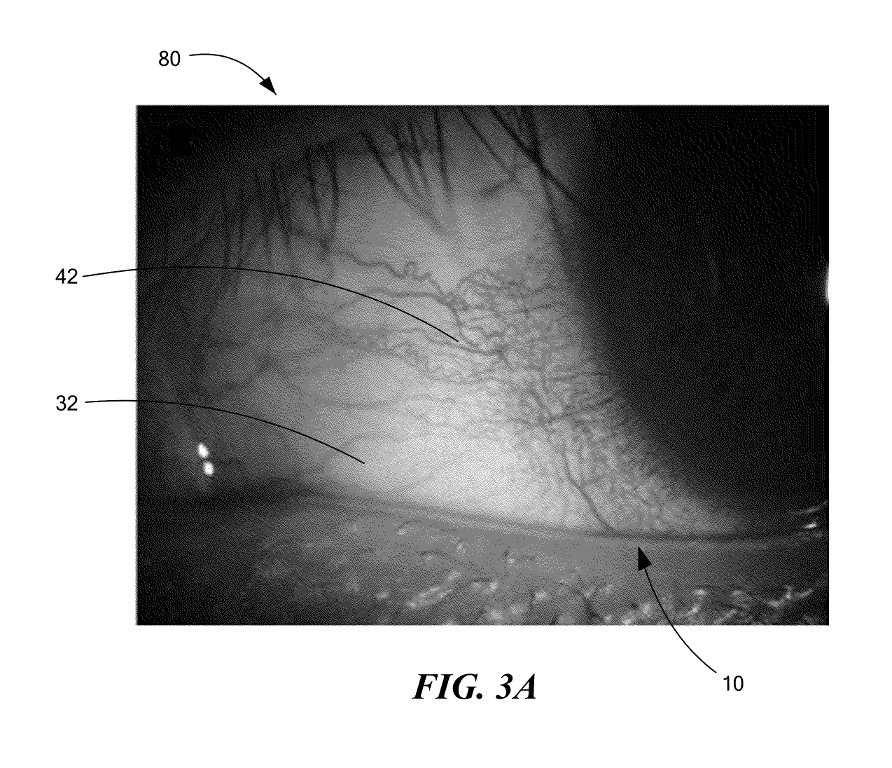

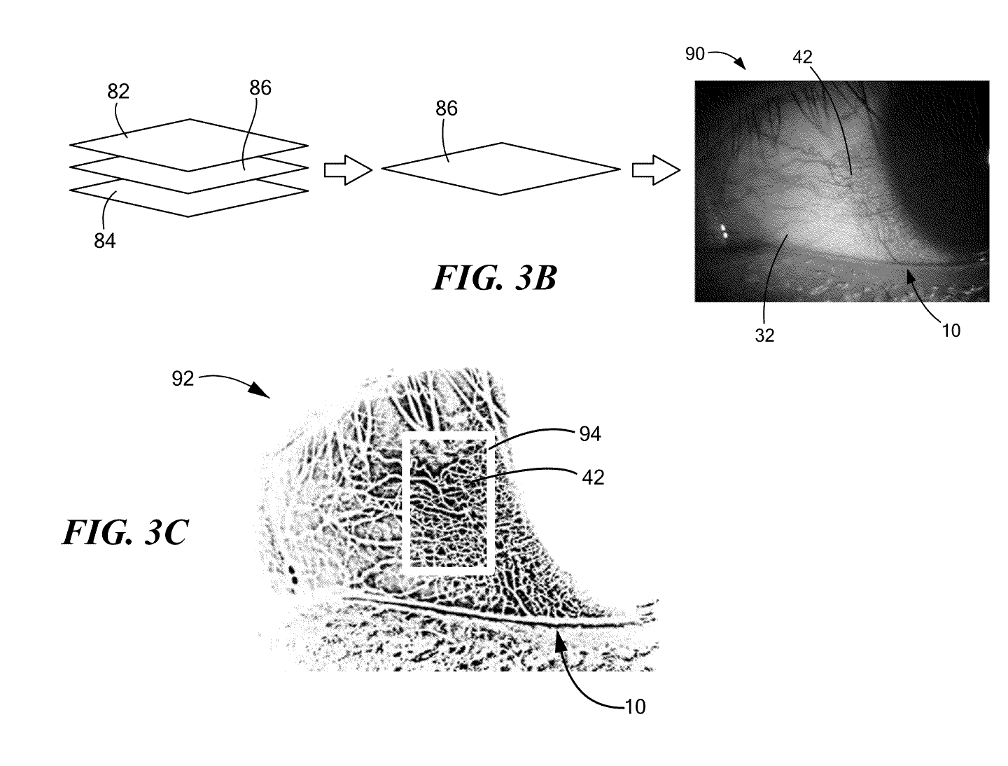

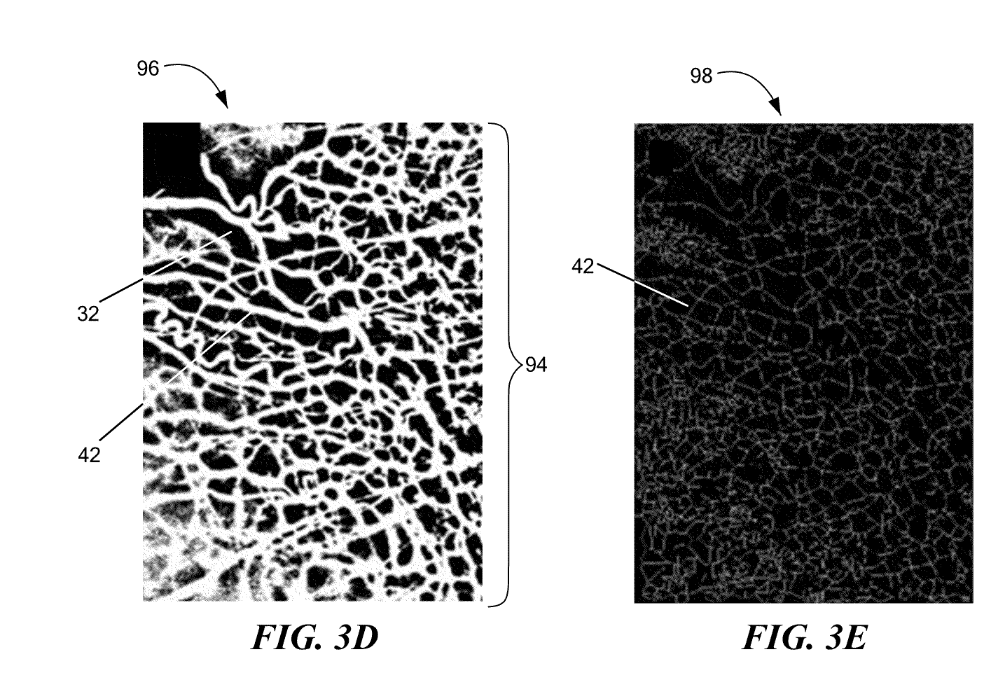

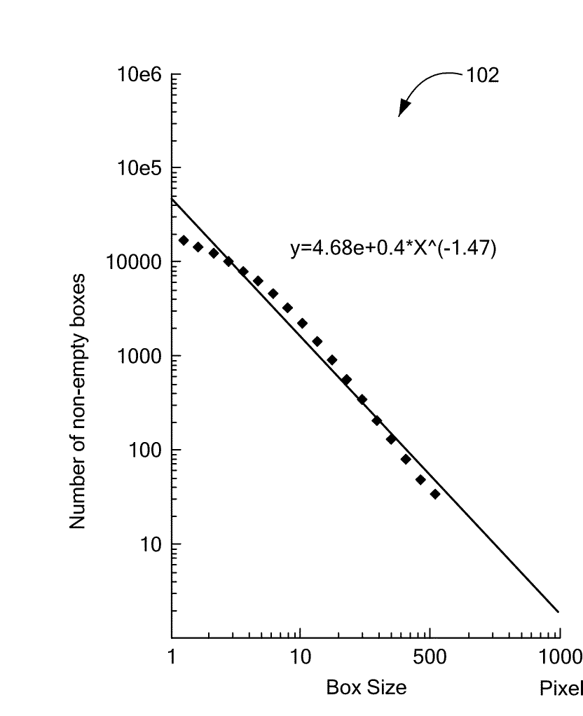

Referring now to FIGS. 3A-3E, a method for obtaining single-shot, high-resolution small vessel perfusion maps of the conjunctival microvasculature is shown. First, a raw image 80 may be obtained using a high-resolution imaging system, such as the system 50 shown and described in FIG. 2. An exemplary raw image 80 is shown in FIG. 3A. Due to the white background of the sclera 32 underneath the capillary bed 42 of the conjunctiva 40, a small vessel perfusion map (also referred to as a “capillary perfusion map” or nCPM) can be generated without any contrast agents (in contrast to currently used methods such as fluorescein angiography) or complicated image processing of multiple images (in contrast to currently used methods such as those using, for example, a Retinal Function Imager (RFI) and associated software). As a non-limiting example, the size of the raw image shown in FIG. 3A may be, for example, 2848×2136 pixels.

In the next step of the method, the channel associated with the green color may be extracted from the raw image 80 using image processing software. For example, as shown in the schematic portion of FIG. 3B, the raw image 80 may include at least a red color channel 82, a blue color channel 84, and a green color channel 86. Extraction of the green channel 86, such as by removal of the red 82 and blue 84 color channels, may improve the contrast between blood vessels 42 and the sclera 32. Extraction of the green channel 86 may result in the creation of a color-adjusted image 90. Images referred to herein may also be called “maps.” Then, the color-adjusted image 90 may be inverted and saved as an 8-bit grayscale image 92 to show the details of small vessel perfusion, as shown in FIG. 3C. The color-adjusted image 90 may be processed with, for example, software such as PHOTOSHOP® (Adobe Systems Incorporated, San Jose, Calif.), to create the inverted grayscale image 92. In the inverted grayscale image 92, which may also be referred to as the nCPM, the vessels 42 of the capillary bed may appear white, whereas the sclera 32 may appear black.

In the next step, a region of interest 94 may then be selected from the inverted grayscale image 92 and enlarged to create an enlarged image 96, as shown in FIG. 3D. As a non-limiting example, the selected region of interest 94 may be as depicted by the white box in FIG. 3C. Finally, the enlarged image 96 of the region of interest 94 may be skeletonized to result in an image 98 suitable for fractal analysis, as shown in FIG. 3E. For example, skeletonizing the image may include reducing anatomical boundaries of each vessel using image processing software so that each vessel 42 may include one or more segments, with each segment having the same diameter (for example, each segment may be the same number of pixels in width along that entirety of the vessel 42). Thus, a vessel 42 may have a uniform diameter, with the diameter of each of a plurality of vessels 42 being the same. This thinning may be accomplished by digitally eroding away pixels from the anatomical vessel boundary while preserving the end points of line segments in the vessel until no more thinning is possible. At this point, a “skeleton” or line network may remain. The line network may then be used for further analysis such as fractal analysis. This may emphasize geometrical and topological properties of the vessel 42 shape that preserves the extent and connectivity of the original anatomical shape of the vessel before skeletonizing the image. All image processing steps and fractal analysis may be performed using one or more types of software executed by one or more processors 70, such as, for example, a processor 70 within the computer 68 of the system 50 shown in FIG. 2.

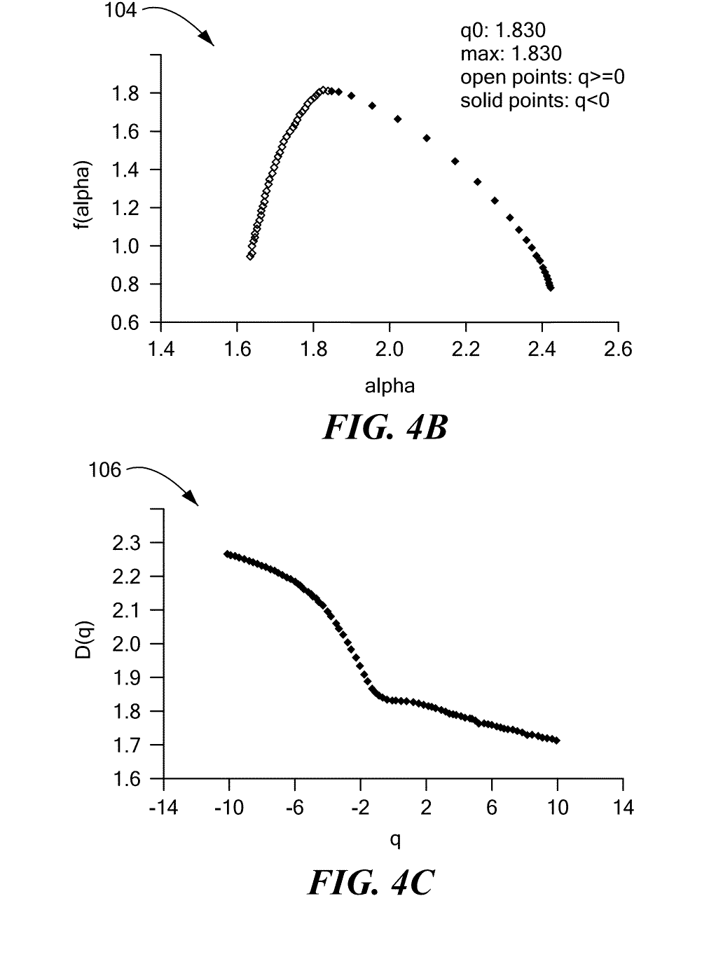

Referring now to FIGS. 4A-4C, non-limiting, exemplary graphs generated using software used for quantitative analysis of the small vessel perfusion map created as a result of the steps shown and described in FIGS. 3A-3E are shown. For example, commercially available software, such as BENOIT™ Pro Fractal Analysis Toolbox (TruSoft Int'l Inc., St. Petersburg, Fla.), may be used to analyze the skeletonized image 98 (for example, as shown in FIG. 3E). The fractal analysis results shown in the graphs of FIGS. 4A and 4B are similar to the results found in literature for normal conjunctival vessel fractal analysis. The graphs 102, 104, 106 shown in FIGS. 4A-4C include a slope of box counting (Db) using mono-fractal analysis that is 1.47 and a result from multifractal analysis (DO) that is 1.83. The normal conjunctival range is 1.0-2.0.

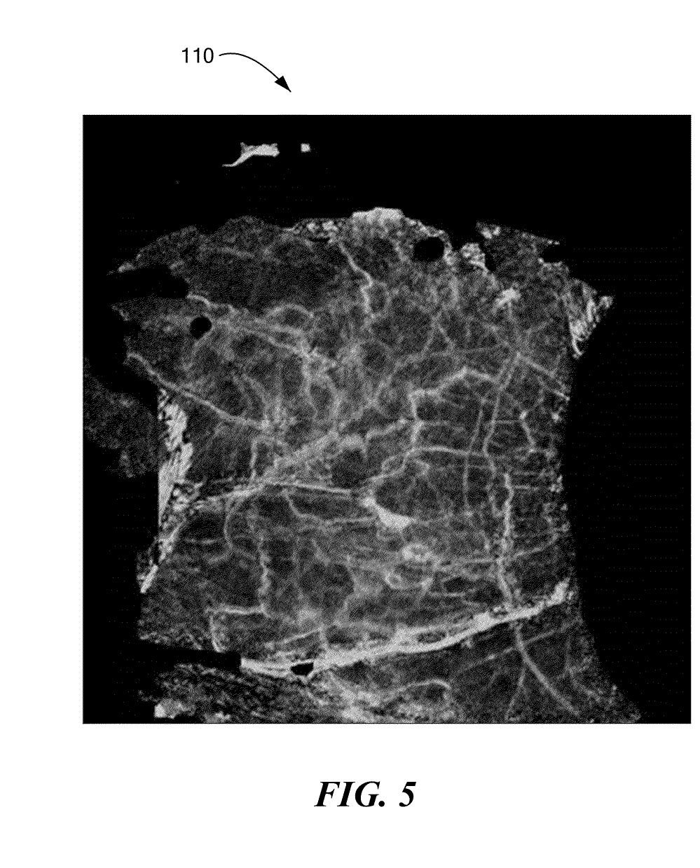

Referring now to FIG. 5, an exemplary capillary perfusion map generated with the Retinal Function Imager (RFI) using currently known methods is shown. This RFI nCPM image 110 is shown as a contrast to the capillary perfusion maps nCPMs obtainable using the present invention (for example, as shown in FIG. 3D). Unlike the images resulting from the method of FIGS. 3A-3E, for example, the RFI nCPM shown in FIG. 5 is the result of multiple images taken and processed using the RFI's custom-built software, and only large vessels can be resolved. Further, the diffuse background of the image of FIG. 5 obscures the details of the small vessels.

The method shown and described in FIGS. 3A-3E may be used to create a capillary perfusion map 92 for determining characteristics such as vascular density, the average vein diameter, and average artery diameter using a quantitative analysis, and a skeletonized image 98, which may be used to assess microvasculature density and complexity using fractal analysis. For example, an exemplary data set for normal subjects is shown in Tables I and II below.

TABLE I
Vein and artery diameter obtained using a perfusion map (n = 2).

| | Diameter (μm) |
|---|---|
| Vein | 15.7 ± 8.79 |
| Artery | 7.0 ± 2.15 |

TABLE II
Fractal analysis (n = 2).

| | nCPMs |
|---|---|
| LX | 1.691 |
| HJ | 1.706 |
| Average | 1.698 |

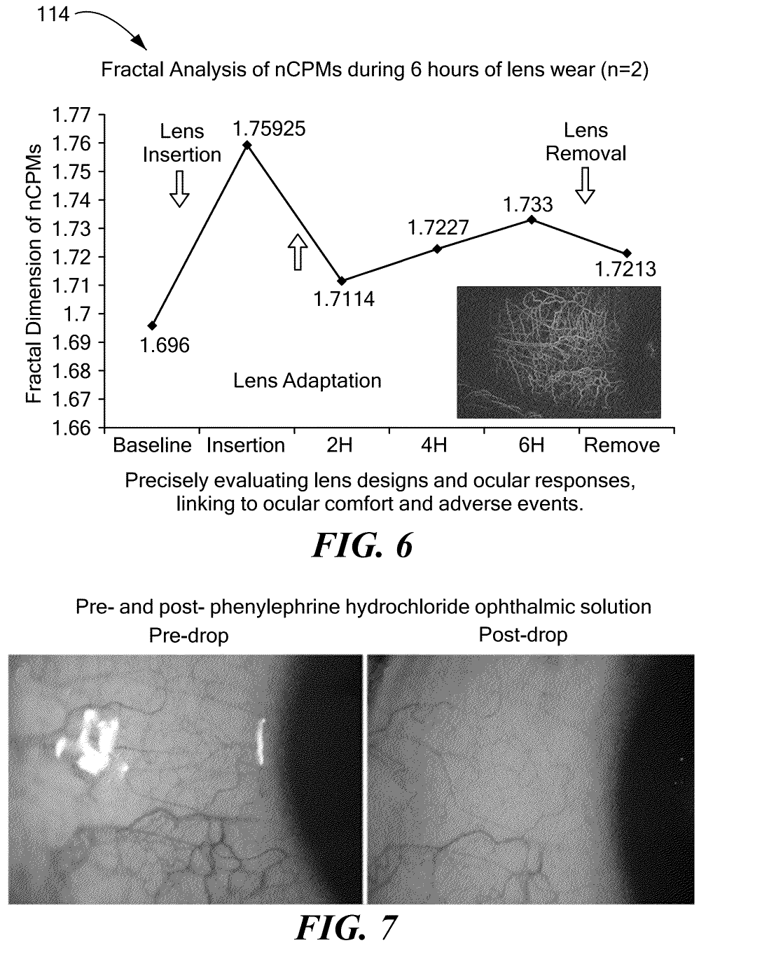

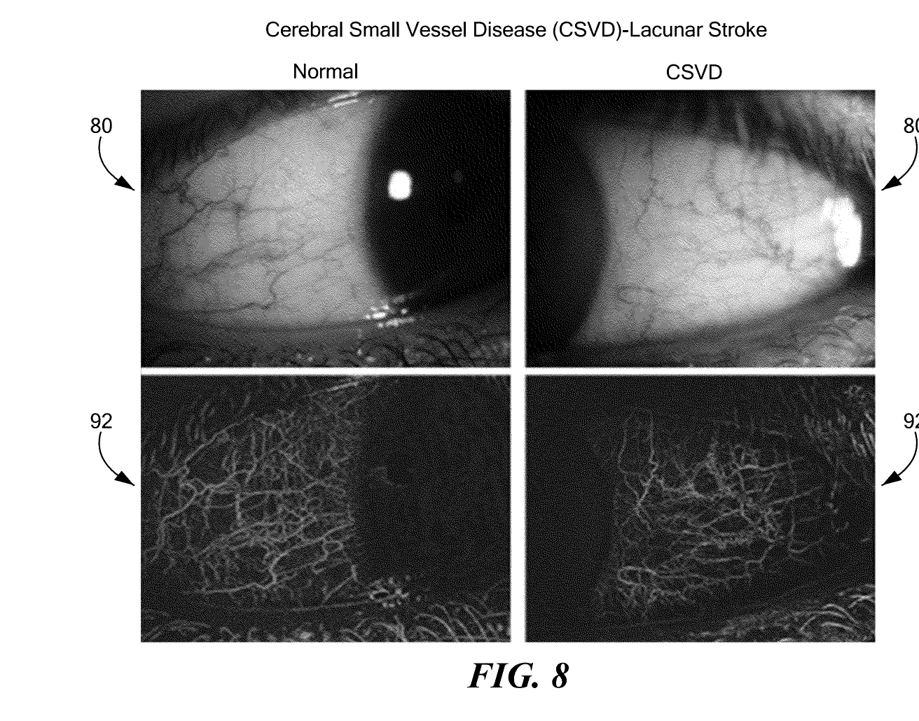

Similarly, nCPMs created using the method of FIGS. 3A-3E may be used to compare these criteria between, for example, “normal” patients (that is, patients without dry eye syndrome) and dry-eye patients (for example, as shown in Table III), between pre-contact lens wear and post-contact lens wear (as shown in the chart 114 of FIG. 6 and in Table IV, such as may be useful for precisely evaluating contact lens designs and ocular responses and linking ocular comfort and adverse events, and for quantifying ocular stress), between pre-exposure and post-exposure to various ophthalmic solutions, such as phenylephrine hydrochloride (as shown in FIG. 7 and Table V, such as may be useful for testing eye whitening drops or artificial tears preservatives), between “normal” patients (that is, patients without multiple sclerosis) and multiple sclerosis patients (as shown in Table VI, such as may be useful in identifying chronic inflammation), between “normal” patients (that is, patients without cerebral small vessel disease) and patients with cerebral small vessel disease (as shown in FIG. 8 and Table VII), and other uses. Therefore, nCPMs obtained using the present invention may be useful for precisely evaluating microvascular perfusion and/or microvascular networks for tracking vascular-related diseases and conditions. For example, diseases such as dry eye, allergies, ocular tumors, corneal transplant rejection, glaucoma, cerebral small vessel disease, hypertension, diabetes, multiple sclerosis, vascular dementia, Parkinson's disease, and Alzheimer's disease, and related conditions such as contact lens wear, ophthalmic drop test, cosmetic eye drop, and artificial tears may be evaluated.

TABLE III
Dry-eye subjects vs. normal subjects (n = 2).

| | | Diameter (μm) | nCPM |
|---|---|---|---|
| Normal | Vein | 15.85 ± 8.79 | 1.698 |
| | Artery | 6.94 ± 2.15 | |
| Dry-eye | Vein | 11.60 ± 5.46 | 1.769 |
| | Artery | 6.25 ± 0.00 | |

TABLE IV
Ocular surface cell stress test/evaluation:
pre- and post-contact lens (CL) wear (n = 2).

| | Diameter (μm) |
|---|---|
| Pre-CL | 13.9 ± 7.3 |
| Post-CL (6 hr) | 20.0 ± 8.3 |

TABLE V
Testing eye whitening drops or artificial
tears: pre-drop and post-drop (n = 5).

| | Diameter (μm) | nCPM |
|---|---|---|
| Pre-drop | 20.00 ± 7.45 | 1.700 |
| Post-drop | 14.58 ± 5.51 | 1.652 |
| Pair t-test | 0.003 | |

TABLE VI
Chronic inflammation as a possible indication of
multiple sclerosis: normal subjects (n = 2)
vs. multiple sclerosis (MS) subjects (n = 4).

| | | Diameter (μm) | nCPM |
|---|---|---|---|
| Normal | Vein | 13.84 ± 7.71 | 1.698 |
| | Artery | 6.63 ± 1.56 | |
| MS | Vein | 15.94 ± 5.95 | 1.732 |
| | Artery | 12.20 ± 2.53 | |

TABLE VII
Cerebral small vessel disease (CSVD), vein hypoperfusion
as a possible indication of lacunar stroke: normal subjects
(n = 2) vs. CSVD subjects (n = 1). “Count”
refers to the number of vessel segments in the analysis.

| | Count | Diameter (μm) | nCPM |
|---|---|---|---|
| Normal | 67 | 13.8 ± 7.7 | 1.698 |
| CSVD | 24 | 15.6 ± 5.7 | 1.671 |

The single-shot, high-resolution perfusion maps of the conjunctival microvasculature disclosed herein may remove human bias and provide diagnostic potential with high specificity and sensitivity. Further, the present invention is more cost effective than systems such as RFI. For example, the RFI method may cost around $150,000 and requires many images to be taken and a complicated semi-automated image processing analysis. Further, the RFI method requires longer computation time and expensive equipment like a fundus camera (which can cost up to fifty times more than a standard slit lamp). In addition, the RFI perfusion method does not produce images that are as clear as the single-shot, high-resolution nCPMs disclosed herein.

From the quantitative image analysis point of view, the detailed structure of conjunctival small vessels observed in high resolution conjunctival perfusion maps generated with a single shot would simplify the methodology to characterize (for example, through quantification and qualification) vascular changes in response to treatment and diseases. As discussed herein, current methods require registration of multiple sequential images to calculate, for example, conjunctival blood vessel diameter. However, microvascular density (a function of disease) could be simply quantified in the single-shot, high-resolution nCPMs to provide a robust measure of tissue oxygenation. Also, fractal dimension, a statistical measure used to characterize the degree of space filling (i.e. complexity) of a vascular network, may be quantified using these single-shot images. This measurement can reflect the efficiency of oxygen and nutrient delivery to the tissue. For example, a normal vasculature is overall more space filling than diseased vasculature, which are spatially heterogeneous with highly avascular as well as densely vascular regions.

The invention of single-shot, high-resolution conjunctival small vessel perfusion will result in a predicting model based on the conjunctival image of systemic and ocular vascular diseases. Further, the technique of the present invention may be used and incorporated into any imaging modality with the capability of taking a single shot of the conjunctiva. This technique may be used to detect abnormalities due to blood or vascular diseases, monitor disease progression, as well as optimize the effect of various pharmacotherapeutics entities in the treatment of vascular diseases. Further, this technique may be used to develop mobile health applications for telemedicine and primary care points. For example, the technique of the present invention may be useful for screening, follow-up, diagnosis, treatment evaluation, patient stratification, clinical trial endpoints, recognizing imaging biomarkers and indicators, risk evolution, community health care, and personal health care.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method for generating a small vessel perfusion map of an ocular conjunctiva, the method comprising:
obtaining a single raw image of at least a portion of microvasculature of the ocular conjunctiva, the raw image including at least a red color channel, a blue color channel, and a green color channel;
removing the red color channel and the blue color channel from the raw image to create a color-adjusted image of the microvasculature; and
inverting the color-adjusted image to create a grayscale inverted image of the microvasculature.

2. The method of claim 1, further comprising skeletonizing the grayscale inverted image of the microvasculature to create a skeletonized image of the microvasculature.

3. The method of claim 2, wherein the microvasculature includes a plurality of vessels, and skeletonizing the grayscale inverted image comprises digitally reducing anatomical boundaries of one or more of the plurality of vessels to one or more linear shapes, each linear shape having a uniform diameter.

4. The method of claim 1, wherein the grayscale inverted image of the microvasculature is cropped to include a region of interest.

5. The method of claim 4, wherein the region of interest is skeletonized and the microvasculature in the skeletonized image is evaluated using fractal analysis.

6. The method of claim 5, wherein the fractal analysis is multifractal analysis.

7. The method of claim 1, wherein at least one of the grayscale inverted image and the skeletonized image is quantitatively analyzed.

8. The method of claim 7, wherein microvascular density is evaluated using at least one of the grayscale inverted image and the skeletonized image.

9. The method of claim 1, further comprising:
determining a threshold value for at least one of conjunctival artery diameter and conjunctival vein diameter;
determining from at least one of the grayscale inverted image and the skeletonized image an average value for at least one of conjunctival artery diameter and conjunctival vein diameter; and
comparing the at least one threshold value to the at least one average value.

10. The method of claim 9, further comprising:
determining the presence of a medical condition based at least in part on the comparison.

11. The method of claim 10, wherein the medical condition is at least one of cerebral small vessel disease, dry eye, chronic inflammation, multiple sclerosis, ocular stress, and hypoperfusion.

12. The method of claim 10, wherein the medical condition is at least one of allergies, ocular tumors, corneal transplant rejection, glaucoma, hypertension, diabetes, vascular dementia, Parkinson's disease, and Alzheimer's disease.

13. The method of claim 9, wherein a response of the ocular conjunctiva to at least one of contact lens wear and the use of an ophthalmic solution is evaluated based at least in part on the comparison.

14. The method of claim 1, wherein the raw image is obtained using a slit lamp or fundus camera.

15. The method of claim 1, wherein the red color channel and the blue color channel are removed using image processing software.

16. A method of evaluating microvasculature of an eye, the method comprising:
obtaining a single raw image of at least a portion of microvasculature of the ocular conjunctiva, the image including at least a red color channel, a blue color channel, and a green color channel;
removing the red color channel and the blue color channel from the raw image to create a color-adjusted image of the microvasculature;
inverting the color-adjusted image to create a grayscale inverted image of the microvasculature; and
skeletonizing the grayscale inverted image of the microvasculature to create a skeletonized image of the microvasculature, the microvasculature including a plurality of vessels, and skeletonizing the grayscale inverted image including digitally reducing one or more of the plurality of vessels to one or more linear shapes, each linear shape having a uniform diameter.

17. A system for generating a small vessel perfusion map of an ocular conjunctiva, the system comprising:
a slit lamp; and
a computer in electrical communication with the slit lamp, the computer including one or more processors programmed to:
obtain a single raw image of at least a portion of microvasculature of the ocular conjunctiva from the slit lamp, the raw image including at least a red color channel, a blue color channel, and a green color channel;
remove the red color channel and the blue color channel to create a grayscale image of the microvasculature;
invert the grayscale image; and
calculate at least one conjunctival microvasculature characteristic.

18. The system of claim 17, wherein the microvasculature includes a plurality of vessels, and the one or more processors are further programmed to:
skeletonize the grayscale inverted image to create a skeletonized image; and
perform a fractal analysis on the skeletonized image.

19. The system of claim 17, wherein the at least one conjunctival microvasculature characteristic is selected from the group consisting of: average conjunctival vein diameter and average conjunctival artery diameter.

20. The system of claim 17, wherein the one or more processors are further programmed to:
determine a threshold value for at the least one conjunctival microvasculature characteristic;
compare the at least one threshold value to the at least one calculated microvasculature characteristic; and
determine the presence of a medical condition based at least in part on the comparison.
